(19) United States
(12) Patent Application Publication
(10) Pub. No.: US 2017/0202811 A1
(43) Pub. Date: Jul. 20, 2017

(54) POROUS DOSAGE COMPOSITIONS AND METHODS OF PRODUCTION

(71) Applicant: Zheng Wang, Bridgewater, NJ (US)

(72) Inventor: Zheng Wang, Bridgewater, NJ (US)

(21) Appl. No.: 15/411,092

(22) Filed: Jan. 20, 2017

Related U.S. Application Data
(60) Provisional application No. 62/280,903, filed on Jan. 20, 2016.

(51) Int. Cl. A61K 31/43; A61K 47/38; A61K 47/36; A61K 47/32; B29C 35/16; B29C 67/00; B33Y 10/00; B33Y 80/00; B29C 35/08; A61K 47/10; A61K 9/00

(52) U.S. Cl. CPC A61K 31/43; A61K 47/10; A61K 47/38; A61K 47/36; A61K 47/32; A61K 9/0056; B29C 67/0059; B33Y 10/00; B33Y 80/00; B29C 35/0805; B29C 35/0866; B29C 35/16; B29C 2035/0827; B29C 2035/0822; B29C 2035/0855; B29C 2035/0872; B29K 2995/006

(57) ABSTRACT

The present invention provides a three-dimensionally printed porous dosage composition and a method of producing the same.

### CROSS-REFERENCE TO RELATED APPLICATIONS

This application claims priority to U.S. Provisional Application No. 62/280,903, filed Jan. 20, 2016, the disclosure of which is incorporated herein by reference.

### FIELD OF THE INVENTION

The present invention relates to a method of producing porous solid dosage composition via 3-D printing.

### BACKGROUND TO THE INVENTION

Standard pharmaceutical dosage forms are administrated orally in the form of tablets and capsules that retain their solid shape under moderate pressure. In general, they are preferable for oral administration with adequate amounts of liquid. There are certain patient populations particularly pediatric and geriatric patients, however, that may have difficulty swallowing solid dosage forms. In some other cases the availability of liquid may be limited. The availability of pediatric or geriatric-friendly dosage forms lags far behind, primarily due to the fact that the overall pediatric market is small and hence a disincentive for research and innovation. Desperate parents of infants and toddlers have to seek help online and exchange ideas on online forums.

Key factors for children's adherence to taking medications include taste, texture and aftertaste of pediatric medications. Medication attributes that promote non-adherence by the child to take the drug are the bitter taste of the drug and frequent dosing. Commonly available pediatric dosage forms are liquid suspension form, oral chewable tablets, suppository and, most recently oral thin films. The oral liquid suspension plays an important role in pediatric formulations and offers convenience to the pediatric patient. However, the unpleasant appearance of a thick suspension deters acceptance by a child as much as unfavorable taste and aftertaste even though accepted by parents themselves. Parents find it difficult to persuade children to take liquid medication.

Common methods of forming fast-disintegrated tablets include freeze-drying techniques, wet granulation and dry granulation. These methods may result in dosage forms with poor mechanical strength and undesirable fragileness. The effectiveness of a freeze-drying process always depends on the physico-chemical parameters of the active substances used. The procedure of replacing the freeze-drying step by conventional drying at room temperature or elevated temperature or drying with microwave radiation is likewise time and energy-consuming, and is also limited to active substances which survive such conditions. An alternative, the so-called Oral Thin Film (OTF) technique uses film-forming hydrophilic polymers that rapidly dissolve upon touching the moist surface of the tongue or buccal cavity. The drug is uniformly distributed within the film matrix and its dosing can be titrated with the child's age and body weight. The OTF vehicle is a two-dimensional thin strip whose size should be preferably smaller than a size of 3 cm by 3 cm for a human dosing. The OTF can be an ideal carrier of an active pharmaceutical ingredient with a relative small dose, preferably less than 50 mg. For a large dose, the film becomes thick and is unable to disintegrate fast enough to retain its advantage as a thin film.

Amoxicillin is the most frequently dispensed prescription medicine in infants (aged 0-23 months) and children (aged 2-11 years). Antibiotics are usually administered either orally or by parenteral route. The taste of an oral antibiotic is a major unresolved issue according to medical practitioners. The oral doses of amoxicillin are high, viz., 200 mg, 250 mg, or 400 mg of amoxicillin as the trihydrate. This high amount of bitter tasting active compound poses a major challenge to the taste-masking efforts especially for dosing amoxicillin in children.

The emerging technology of 3D printing is the process of making a three-dimensional object, in which successive layers of materials are laid down under computer control. The resulting objects can be of almost any shape or geometry and can be produced from a 3D model or other electronic data sources. It is promising that 3D printing solutions would allow medical manufacturers and doctors to create parts for patient care and advanced experimental work. The most common 3D printer utilizes the fused filament fabrication (FFF) technology where a machine extrudes a thermoplastic material filament and positions into layer by layer controlled by a computer program.

U.S. Pat. No. 6,471,992 teaches a 3D printing technique to produce a rapidly dispersing dosage form. U.S. Pat. No. 6,280,771 describes that dosage forms prepared by a 3D printing provide release of medicament in multiple phases. The process described in the patents results in a weak bonding which attributes to its fast dispersing upon contacting moisture, however the weak bonding also leads to poor mechanical strength and have insufficient holding capacity for active ingredients.

A need exists for a new dosage form that rapidly disintegrates and dissolves in the mouth to release the drug actives for oral mucosal and intra-gastric absorption, without chewing and intake of water. In particular, such a dosage form will provide a significant benefit for pediatric and geriatric patients.

### SUMMARY OF THE INVENTION

The present invention met such a need. A novel dosage unit and its method of manufacture and use are provided in the present invention. In particular, a direct-write 3D (DW3D) printing process is developed to produce an oral disintegrated drug unit of designed shape and geometry with desirable mechanical strength and active-holding capacity.

A direct-write 3D (DW3D) technique employs a computer-controlled translation stage, which moves an ink-deposition nozzle, to create materials with designed architecture and composition. Under applied pressure, an ink filament undergoes capillary shear flow inside the micronozzle, relaxes its stresses upon exiting the nozzle. The materials then form rigid structures as the highly volatile solvents (dichloromethane (DCM) or chloroform) evaporate immediately. However, the common-used solvent DCM is highly volatile but toxic. It may be retained within the scaffold. The DW3D technique requires specific viscoelastic and rheological properties. The ink viscosity cannot be high for extrusion through a capillary nozzle under applied pressure. Furthermore, the rigidity of the extruded material must be increased in order to retain its shape.

The present invention overcomes the drawback of the conventional processes. A water-containing mixture prepared for 3-D printing not only eliminates the toxicity issue related to halogenated solvents but also delivers a solid dosage in desired form and mechanical strength. The printer can have a single extruder head or multiple extruder heads to create materials with controlled architecture and composition. Other suitable devices include for example those with a continuous jet stream print head which provides for a fluid that is pressure driven through a small orifice.

### BRIEF DESCRIPTION OF THE DRAWINGS

FIG. 1 shows a schematic diagram of the Direct-Writing 3D (DR3D) printing system for the preparation of a porous multi-surface drug vehicle.

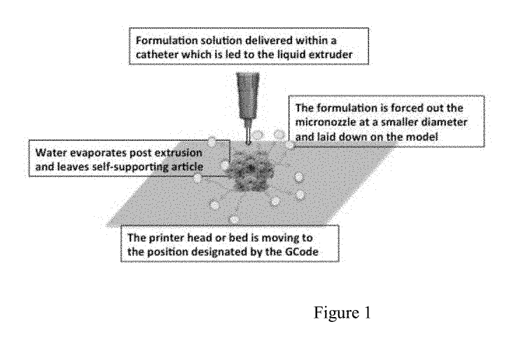

FIG. 2 shows a plot of the mean plasma concentration versus time curves for Test Article (TA: Amoxicillin Product) and Reference Article (RA: Dispersible Tablets).

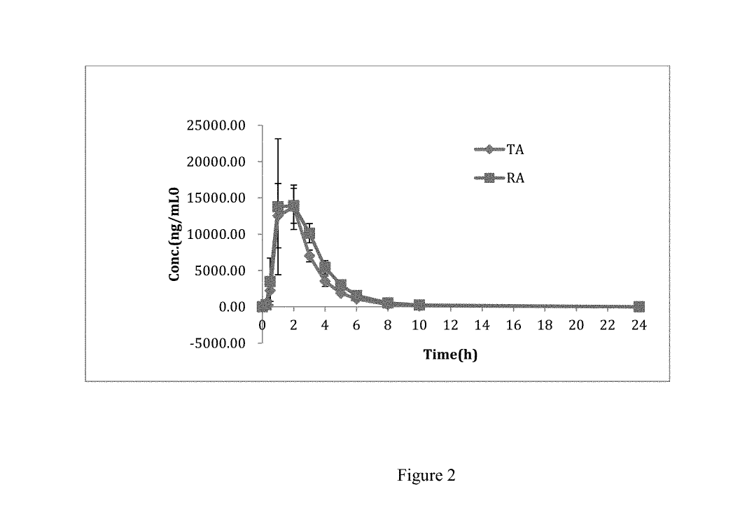

### DETAILED DESCRIPTION OF THE INVENTION

The present invention relates to an oral dosage form, method of oral disintegrated delivery form of therapeutic agents, and production of the dosage form. In contrast to the conventional approaches such as lyophilization processes used in the preparation of fast-disintegrating solid dosage forms, which are laborious, expensive and energy intensive, the present invention uses a highly economical process and novel computer-assisted 3-dimensional (3D) printing for the manufacture of solid highly porous fast-disintegrating dosage forms. For patients having problems ingesting standard oral dosage forms, in particular small children and elderly patients, it is a welcome simplification when the pharmaceutical compound is dissolved immediately on ingestion without additional liquid.

In order to more clearly and concisely describe the subject matter of the claims, the following definitions are intended to provide guidance as to the meaning of terms used herein.

The articles “a” and “an” as used herein mean “one or more” or “at least one,” unless otherwise indicated. That is, reference to any element of the present invention by the indefinite article “a” or “an” does not exclude the possibility that more than one of the element is present.

The term “about” means the referenced numeric indication plus or minus 10% of that referenced numeric indication.

The term “drug” includes any drug appropriate for administration via the oral route. The term “drug” also includes those pharmaceutical entities that are poorly absorbed via the traditional oral route including hydrophilic drugs.

The term “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder.

The term “subject” refers to a human or animal species.

The term “bioerodible” of “bioerodibility” refers to a water erodibility of a substance in a subject, ranging from negligible to completely water erodible. The substance may readily dissolve in water or may only partially dissolve in water with difficulty over a long period of time. Furthermore, the substance may exhibit a differing erodibility in body fluids compared with water because of the more complex nature of body fluids. For example, a substance that is negligibly erodible in water may show an erodibility in body fluids that is slight to moderate. However, in other instances, the erodibility in water and body fluid may be approximately the same. A bioerodible substance may exhibit one or more properties. For example, a bioerodible substance may facilitate the dosage unit to stay in close contact with a mucosal surface without substantial movement in the mouth. Accordingly, in some embodiments bioerodibility and bioadhesiveness are both desirable properties of the bioerodible substance of an oral dosage unit, which could be in the form of a film, a single layer or a multilayer. A water soluble polymer may serve as a bioerodible substance in the oral dosage unit. Non-limiting examples of bioerodible substances include sodium carboxymethyl cellulose (NaCMC), hydroxypropylmethyl cellulose (HPMC), polyvinylpyrrolidone (PVP), hydroethylmethyl cellulose (HEMP), pullulan, modified starch, polyethylene oxide, polyacrylate, and combinations thereof.

The term “dosage form” or “dosage composition” refers to a drug product that contains a therapeutic agent calculated to produce a desired effect. “Unit dosage form” refers to a physically discrete unit suitable as a unitary dosage for a human or an animal.

The term “excipient” refers to an inert substance combined with an therapeutic agent to prepare a convenient dosage form and vehicle for delivering the therapeutic agent.

The term “a therapeutically effective amount” refers to an amount of a therapeutic agent or a drug that elicits a therapeutically useful response in treating an existing medical and/or preventing or delaying the onset of a medical condition from occurring in an animal, preferably a mammal, most preferably a human.

The term “transmucosal,” refers to any route of administration via a mucosal membrane or surface. Examples include, but are not limited to, buccal, sublingual, nasal, vaginal, and rectal. In some embodiments, the term “transmucosal delivery” refers to mucosal administration via the oral mucosa, e.g., buccal and/or sublingual.

The term “permeation enhancer” refers to a natural or synthetic molecule which facilitates the absorption of a drug or an active agent through a mucosal surface. For example, phospholipi ds, including phosphatidylcholines, phosphatidylethanolamines, and phosphatidylinositols, can serve as a permeation enhancer. Specific examples of phospholids also include 1,2-dimyristoyl-sn-glycero-3-phosphocholine, 1,2-dioleoyl-sn-glycero-3-phosphocholine, 1,2-di stearoyl-sn-glycero-3-phosphocholine.

The term “surfactant” refers to materials which preferably orient toward an interface, classes of surfactants including nonionic surfactants, anionic surfactants, cationic surfactants, amphoteric surfactants, zwitterionic surfactants, and mixtures thereof.

The term “therapeutic agent” or “pharmaceutically active agent” is used herein to refer to a substance or formulation or combination of substances or formulations of matter which, when administered to a subject (human or animal) induces a desired pharmacologic and/or physiologic effect by local and/or systemic action.

An aspect the invention provides a three-dimensionally printed porous dosage composition. The composition contains an active agent, a solublizing agent, a structuring agent, a sweetening agent and a taste-masking agent. It is further characterized by having pre-designed void space ranging between 10% to 80% of the total volume of the composition.

The dosage forms or compositions of the present invention can be in the form of, for example, small disks, stars, rods or sheets, but can also be termed tablets, although these products do not represent tablets in the usual sense obtainable by compression. Other exemplary forms include spherical or ellipsoid shapes, rods, granules, blocks, cubes with rounded edges.

Various parameters such as size and shape of the void space of the dosage can be readily controlled by the 3D printing. In exemplary embodiments, the void space accounts for about 10%, 20%, 30%, 40%, 50%, 60%, 70% and 80% of the dosage form. The void space can be created by printing according to a pre-designed pattern. In addition, by adjusting the amount of the solvent and the removal of the solvent, the void space can also be controlled.

The dosage composition described herein generally has a lower density than drug products manufactured with conventional processes. The density of the dosage composition of the present invention ranges from about 15 to about 800 mg/cm³. Exemplary ranges of density include about 15 to about 600 mg/cm³, about 15 to about 400 mg/cm³, about 15 to about 300 mg/cm³, about 15 to about 200 mg/cm³, and about 15 to about 100 mg/cm³.

The low density in combination with the void space contributes to faster disintegration of the composition. In some embodiments, the dosage form or composition disintegrates substantially in the mouth of a subject within 5-10 minutes. In some embodiments, the unit dosage form or composition disintegrates substantially in the mouth of a subject in less than 8, 7, 6, 5, 4, or 3 minutes.

The active agent in the dosage composition is preferably a pharmaceutical but may also be, for example, Caffeine and Caffeine salt compounds, vitamins, minerals or dietary supplements. Pharmaceuticals may include, without limitation, antacids, analgesics, anti-inflammatory agents, antibiotics, laxatives, anorexics, anti-asthmatics, diuretics, antiflatulents, antimigraine agents, anti-arrhythmic agents, antispasmodics, sedatives, antihyperactive agents, tranquilizers, antihistamines, decongestants, beta-blockers, coronary vasodilators, bronchodilators, muscle relaxants, anticoagulants, antileptic agents, anti-emetics, hypotensives, sympathomimetic agents, expectorants, oral antidiabetic agents, hormones and combinations thereof.

By way of example, amongst the pharmaceutically active substances, the active agent can be an antibiotic. β-lactam antibiotics are a broad class of antibiotics that include any antibiotic agent that contains a β-lactam nucleus in its molecular structure. β-lactam antibiotics include, but are not limited to, penicillins, penicillin derivatives, cephalosporins, monobactams, carbapenems, and β-lactamase inhibitors. Examples of β-lactam antibiotics include benzathine penicillin, benzylpenicillin (penicillin G), phenoxymethylpenicillin (penicillin V), procaine penicillin, oxacillin, methicillin, nafcillin, cloxacillin, dicloxacillin, flucloxacillin, temocillin, amoxycillin, ampicillin, azlocillin, carbenixillin, mezlocillin, and piperacillin.

The active agent in the dosage composition generally ranges from about 5% to about 85%, all sub-ranges and sub-values included, by weight. Examples of the percentage weight include about 5%, about 10%, about 15%, about 20%, about 25%, about 30%, about 35%, about 40%, about 45%, about 50%, about 55%, about 60%, about 65%, about 70% and about 80%.

Other active agents suitable for the present invention include for example anti-inflammatory and anti-rheumatic agents, analgesics, tranquilizers, cardiovascular agents and cerebral vasodilators, cerebral protectors, antispasmodic and antisecretory agents, antiasthmatic agents, therapeutic agents for diseases of the gastrointestinal tract, hepatic protectors, hormones, contraceptives, medicaments intended for the treatment of allergies, vaccines, vitamins or peptides, polypeptides or proteins.

The dosage composition can include water-soluble hydrophilic polymer carrier, pharmaceutical active ingredient, solubilizers, oral absorption enhancers, taste-masking agents, emulsifier, plasticizer, buffering agent, coloring agent, and other necessary excipients.

Various solubilizing agent can be used to prepare the composition of the present invention. In some embodiments, the agent is a PEG. The molecular weight of the PEG may vary and examples include PEG 20, PEG 200, PEG 400, and PEG 3000. In some embodiments, the solubilizing agent or the composition is substantially free from a chlorinated solvent (e.g. methylene chloride). The solubilizing agent in the composition ranges for example from about 5% to about 25%, from about 5% to about 20%, from about 5% to about 15%, from about 5% to about 10%, from about 10% to about 15%, and from about 8% to about 10%.

Examples of taste-masking agents include sucralose, aspartame, Magnasweet, and/or other natural herb sweet extracts such as Chrysanthemum lavandulifolium (Fisch.ex Trautv.).

For purposes of oral delivery, the dosage form can be applied on lingual, sub-lingual, buccal, gingival, and palatal surfaces. On oral application through the buccal cavity, for example when putting on the moist tongue, they rapidly take up water from saliva, mollify, disintegrate immediately, or are easily chewed or crushed with the tongue. Even if the dosage form is not completely dissolved there will be a pasty mass easily swallowable. Accordingly, the present invention also provides a method of administering the dosage composition described herein by contacting the composition with a lingual, sub-lingual, buccal, gingival, and palatal surface of a subject in need.

In connection with the dosage form described herein, the present invention also provides a method of treating disease by administering to a subject in need an effective amount of the dosage form or composition of the present invention. The pharmaceutically active agent is selected from the group consisting of δ-lactam antibiotics, Caffeine, Caffeine salt compounds, vitamins, antibiotics, antacids, analgesics, anti-inflammatory agents, laxatives, anorexics, anti-asthmatics, diuretics, antiflatulents, antimigraine agents, anti-arrhythmic agents, antispasmodics, sedatives, antihyperactive agents, tranquilizers, antihistamines, decongestants, beta-blockers, coronary vasodilators, bronchodilators, muscle relaxants, anticoagulants, antileptic agents, anti-emetics, hypotensives, sympathomimetic agents, expectorants, oral antidiabetic agents, anti-inflammatory and anti-rheumatic agent, tranquilizers, cardiovascular agents and cerebral vasodilators, cerebral protectors, antispasmodic, antisecretory agents, hepatic protectors, hormones, contraceptives, polypeptides and proteins, medicaments intended for the treatment of ADHD, allergies, vaccines, erectile dysfunction, and combinations thereof.

The dosage compositions of the present invention can be applied to the treatment of various diseases, including but not limited to, autoimmune/autoinflammatory diseases, bacterial infections, behavioral and mental disorders, congenital and genetic diseases, digestive diseases, connective tissue diseases, blood diseases, ear/nose/throat diseases, endocrine diseases, environmental diseases, eye diseases, male/female reproductive diseases, fungal infections, heart diseases, hereditary cancer syndromes, immune system diseases, kidney and urinary diseases, lung diseases, metabolic disorders, mouth diseases, musculoskeletal diseases, myelodysplastic syndromes, nervous system diseases, nutritional diseases, parasitic diseases, rare cancers, skin diseases, viral infections.

The dosage unit may be produced by the direct-write assembly of the 3D printing technique that employs a computer-controlled translation stage, which moves a formulation-deposition nozzle, to create materials with controlled architecture and composition. This Direct-Writing 3D (DW3D) system requires specific rheological and viscoelastic properties of the printed formulation solution.

In an exemplary embodiment, a mixture or a formulation solution is delivered within a catheter led to the liquid extruder. The formulation is then forced out the micronozzle at a smaller diameter and laid down on the position designed by the GCode. A GCode file of a 3D porous structure (snowflakes, for an example) was generated in order to print prototypes for evaluation. For successful direct writing of 3D freeform structures, the formulation solution has to be evaluated to ensure proper extrusion ink rheological behavior. The speed of the extrusion nozzle and the applied pressure of the formulation solution must be tailored to achieve the desired material linear flow rate.

In exemplary embodiments, the orifice diameter of the single or multiple extruder heads ranges from 0.10 mm to 1mm. The extruder speed ranges from about 100-5000 steps/mm. However, these parameters can certainly vary beyond the range depending on the particular composition to be prepared.

Product curing conditions, trace-binding, and other process parameters will be monitored and optimized during this investigation. An additional air-circulation chamber will be built to provide extra air-flow to the deposit model area to expedite the material binding and curing process. Partial drying process is performed by applying convection heat, infra-red, UV, microwave, freeze-drying, direct-ion technology, or any other drying techniques to said deposited structure.

These oral dosage form or composition prepared according to the methods of the present invention have an extended shelf life. They are easy to handle and non-tacky before administration so as to avoid disintegration prior to use and are conveniently packaged for shelf life, ease of storage and distribution.

In further exemplary embodiments of the production process, a mixture, which can be for example a suspension, a solution or an emulsion, is prepared and loaded to the printer for printing. Preferably, the mixture is homogeneous. In some embodiments, water is a solvent used to prepare the mixture as illustrated in the production of Examples 1-6 of Table 1. Water content in the mixture can range from 20% to 85%. The amount of water in the mixture can vary and determine the viscoelastic properties of the mixture solution or suspension. A high water content can be more achievable for extrusion because its lower viscosity makes it easier to flow through the nozzle orifice, however the resulting 3D-structure generally requires extended or complete drying in order to have a freehold form structure. A low water content formulation can be more desirable in some cases for the purpose of 3D dosage unit manufacturing where the drying process can become easily achievable. In some embodiments, water accounts for about 20%, about 30%, about 40%, about 50%, about 60%, about 70%, or about 80% of the total weight of the mixture for printing. In some embodiments, water accounts for about 10-30%, about 20-30%, about 30-40%, about 40-50%, about 50-60%, about 60-70%, about 70-80%, or about 80-90% of the total weight of the mixture before being subject to printing. In some embodiments, the mixture is substantially free from halogenated solvent. In some embodiments, the solvent in the mixture is substantially water. In some embodiments, the mixture contains one or more additional non-halogenated organic solvents besides water.

In further exemplary embodiments, a method of production includes the steps of: preparing a solution or suspension of an active ingredient; mix the solution or suspension with a permeation enhancer and/or taste-masking agents, or more excipients; optionally emulsify the resulting mixture and/or reducing the size the particles in the mixture; optionally combing the mixture with other excipients; printing the mixture to a supporting material mounted on the printing bed, and removing the excess solvent (water) to obtain a solid dosage form.

Without being bound to any particular concept, the following methods A and B are for illustration of the preparation of the oral dosage unit of the present invention.

Method A (DR3D Printing):

Preparing a homogenous mixture of drug active, taste masking agent, bioerodible substance, permeation enhancer, and one or more excipients;

Printing the mixture to a supporting material mounted on the printing bed; and

Drying the film to remove excess solvent if needed.

Method B (Film Coating):

Preparing a homogenous mixture of drug active, taste masking agent, bioerodible substance, permeation enhancer, and one or more excipients;

Coating the mixture to a supporting material mounted; and

Drying the film to remove excess solvent if needed.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

For patients with difficulties controlling their swallowing, the present invention also offers the advantage of adjusting the dissolution rate of the dosage unit on the mucosal surface so that the agent will not be released too fast and swallowed subsequently. As a result of such controlled release, mucosal uptake of the agent will be enhanced.

### EXAMPLES

The following examples 1-7 relate to formulations and preparation of amoxicillin oral disintegrated vehicle.

TABLE 1
amoxicillin compositions

| | Example | Example | Example | Example | Example | Example | Example |
|---|---|---|---|---|---|---|---|
| | 1 | 2 | 3 | 4 | 5 | 6 | 7 |
| Amoxicillin | 50.0% | 50.0% | 50.0% | 50.0% | 50.0% | 50.0% | 50.0% |
| trihydrate | | | | | | | |
| PEG400 | 10.0% | 10.0% | 10.0% | 10.0% | 10.0% | 10.0% | 10.0% |
| Bitterness | 0.0% | 0.0% | 2.0% | 2.0% | 2.0% | 0.01% | 0.20% |
| reducer#2218 | | | | | | | |
| Sucralose | 0.0% | 1.0% | 1.0% | 0.01% | 0.1% | 0.2% | 0.2% |
| Pullulan | 38.95% | 37.95% | 35.95% | 36.96% | 36.85% | 38.75% | 38.55% |
| TiO2 | 1.0% | 1.0% | 1.0% | 1.0% | 1.0% | 1.0% | 1.0% |
| FD&C Blue#1 | 0.050% | 0.050% | 0.050% | 0.050% | 0.050% | 0.050% | 0.050% |
| SUM | 100.0% | 100.0% | 100.0% | 100.0% | 100.0% | 100.0% | 100.0% |

In embodiments of the invention, the taste-masking agents include sucralose, aspartame, Magnasweet, and/or other natural herb sweet extracts such as Chrysanthemum lavandulifolium (Fisch.ex Trautv.). In Examples 1-7, one major sweetener in Bitterness reducer#2218 is the extract of Chrysanthemum lavandulifolium.

Preliminary taste of Amoxicillin prototypes of Examples 1-7 was evaluated by the time intensity method. Amoxicillin dose unit containing 125 mg of amoxicillin trihydrate was evaluated for prototypes of Formulations 1-7 tableted in Examples 1-7. All prototypes were prepared using the Method B (Film Coating). A single unit was held underneath the tongue and dissolved in a few minutes without drinking water. Immediate bitterness was recorded within one (1) minute after administration. After-taste was recorded 15 minutes after administration. Subjects were instructed not to swallow the dissolving materials. Bitterness levels were recorded according to the numeric system below:
Bitter
1=none
2=very slight (some bitterness)
3=slight (noticeable bitterness)
4=moderate (intense bitterness)
5=high (very intense bitterness)

TABLE 2
Taste Evaluation

| Taste | Example | Example | Example | Example | Example | Example | Example |
|---|---|---|---|---|---|---|---|
| Evaluation | 1 | 2 | 3 | 4 | 5 | 6 | 7 |
| Immediate | 5 | 1 | 1 | 4 | 2 | 1 | 1 |
| bitterness | | | | | | | |
| After taste | 5 | 4 | 1 | 2 | 1 | 3 | 1 |
| (bitterness) | | | | | | | |

The key variables of Examples 1-7 are the sweet agents of sucralose and the Bitterness reducer#2218 (extract of Chrysanthemum lavandulifolium). The taste evaluation results in the above table suggest that sucralose is sufficient to mask the immediate taste of the oral amoxicillin article, however the Bitterness Reducer #2218 plays a significant role in reducing the after-taste of the product.

The following Examples 8-13 relate formulations and preparation of Amoxicillin oral disintegrated articles using the DR3D Printing Technique.

TABLE 3
amoxicillin compositions

| | Example | Example | Example | Example | Example | Example |
|---|---|---|---|---|---|---|
| | 8 | 9 | 10 | 11 | 12 | 13 |
| Amoxicillin trihydrate | 83.3% | 83.3% | 75.8% | 75.8% | 75.8% | 75.8% |
| Carboxymethylcellulose | 1.0% | 1.0% | 1.0% | 1.0% | | |
| sodium (CMC Na) | | | | | | |
| PEG400 | 9.0% | 9.0% | 9.0% | 9.0% | 8.0% | 10.0% |
| Bitterness reducer#2218 | 0.2% | | 0.2% | | | |
| Sucralose | 0.2% | 0.2% | 0.2% | 0.2% | 0.2% | 0.2% |
| Pullulan | 6.2% | 6.4% | 13.8% | 11.5% | | |
| Gelatine | | | | 2.5% | | 3.0% |
| Hydroxypropylmethyl- | | | | | 8.0% | |
| cellulose (HPMC) | | | | | | |
| Methylcellulose | | | | | 8.0% | |
| Polyvinylpyrrolidone | | | | | | 11.0% |
| (PVP) | | | | | | |
| FD&C Blue#1 | 0.025% | 0.025% | 0.025% | 0.025% | 0.025% | 0.025% |
| SUM | 100.0% | 100.0% | 100.0% | 100.0% | 100.0% | 100.0% |

The general procedures for preparing or manufacturing amoxicillin oral disintegrated article are described briefly below. A formulation mixture mass containing a homogenous mixture of ingredients in the amount indicated in Table 3 was prepared with addition of water as solvent (approximately water content 20%-85%). The prepared formulation solution was de-gassed in a vacuum mixer and delivered within a catheter led to the liquid extruder. The formulation is then forced out the micronozzle at a smaller diameter and laid down on the position designed by the GCode. A GCode file of a 3D porous structure (snowflakes, for an example) was generated in order to print prototypes for evaluation. For successful direct writing of 3D freeform structures, the formulation solution has to be evaluated to ensure proper extrusion ink rheological behavior. The speed of the extrusion nozzle and the applied pressure of the formulation solution must be tailored to achieve the desired material linear flow rate.

Product curing conditions, trace-binding, and other process parameters are monitored and optimized. An additional air-circulation chamber was built to provide extra air-flow to the deposit model area to expedite the material binding and curing process. Partial drying process can be achieved by applying convection heat, infra-red, UV, microwave, freeze-drying, direction technology, or any other drying techniques to said deposited structure.

Pharmacokinetics of Amoxicillin Oral Disintegrated Vehicle (Formulation #P64) in Male Beagle Dogs Following Oral Administration

An animal PK study of Amoxicillin Product was conducted by the Medicilon Preclinical Research-Shanghai LLC in Shanghai, China. The purpose of this study was to determine the pharmacokinetic (PK) profiles of amoxicillin trihydrate via single oral or buccal administration in beagle dogs.

TABLE 4
Animal PK

| | Animal | Test | Dose Level | Route of | |
|---|---|---|---|---|---|
| Group | number Male | Articles | (mg/animal) | Dosing | Collection |
| 1 | 3 | TA | 250 mg | Buccal | Plasma |
| 2 | 3 | RA | 250 mg | PO | Plasma |

Clinical Observations

No obvious abnormal observations were noted, pre- and post-dose with dogs. FIGS. 4 shows a comparison of profiles of mean plasma concentration versus time of ta and ra in beagle dogs.

TABLE 5
Selected Pharmacokinetic Parameters of TA in Beagle
Dogs Following Single Buccal Administration.

| Animal | T_{1/2} | Tₘₐₓ | Cₘₐₓ | AUC₀₋ₜ | AUC_{0-∞} | MRT_{0-∞} |
|---|---|---|---|---|---|---|
| Number | (h) | (h) | (ng/mL) | (ng · h/mL) | (ng · h/mL) | (h) |
| 101 | 3.44 | 1.00 | 14116.97 | 38273.78 | 38332.24 | 2.77 |
| 102 | 3.49 | 2.00 | 12766.15 | 37019.14 | 37086.89 | 3.18 |
| 103 | 4.62 | 2.00 | 17132.26 | 45272.55 | 45411.56 | 2.66 |
| Mean | 3.85 | 1.67 | 14671.79 | 40188.49 | 40276.89 | 2.87 |
| SD | 0.67 | 0.58 | 2235.31 | 4447.39 | 4490.13 | 0.28 |

TABLE 6
Selected Pharmacokinetics Parameters of RA in Beagle
Dogs Following Single Oral Administration.

| Animal | T_{1/2} | Tₘₐₓ | Cₘₐₓ | AUC₀₋ₜ | AUC_{0-∞} | MRT_{0-∞} |
|---|---|---|---|---|---|---|
| Number | (h) | (h) | (ng/mL) | (ng · h/mL) | (ng · h/mL) | (h) |
| 201 | 2.58 | 2.00 | 15694.87 | 48015.81 | 48033.73 | 2.86 |
| 202 | 2.59 | 2.00 | 11175.68 | 35750.59 | 35767.73 | 3.31 |
| 203 | 2.69 | 1.00 | 23745.90 | 65328.12 | 65378.36 | 3.01 |
| Mean | 2.62 | 1.67 | 16872.15 | 49698.18 | 49726.60 | 3.06 |
| SD | 0.06 | 0.58 | 6367.27 | 14860.36 | 14877.73 | 0.23 |

Conclusions

No obvious abnormal observations were noted, pre- and post-dose with dogs.

Following single buccal administration of TA in dogs, the mean half-life of TA was 3.85 h. The mean value of Cₘₐₓ and AUC_{(0-∞)} were 14671.79 ng/mL and 40188.49 ng/mL*h, respectively.

Following single oral administration of RA in dogs, the mean half-life of RA was 2.62 h. The mean value of Cₘₐₓ and AUC_{(0-∞)} were 16872.15 ng/mL and 49726.60 ng/mL*h, respectively.

This preliminary animal PK study suggests that Amoxicillin Product (Formula #64) is a suitable alternative delivery system with comparable bioavailability (Cₘₐₓ, AUC(0→∞) and longer half-life (T1/2, 3.85 hours-TA versus 2.62 hours-RA) to its reference product of dispersible tablets.

1. A method of preparing a porous dosage composition, comprising:
(a) loading to a printer a mixture comprising a pharmaceutically active agent and water, wherein the weight percentage of the water in the mixture ranges from about 15% to 85%;
(b) printing the mixture according to a pre-designed pattern; and
(c) removing the water to obtain the porous solid dosage composition.

2. The method of claim 1, wherein the weight percentage of water in the mixture ranges from about 20% to 50%.

3. The method of claim 1, wherein the weight percentage of water in the mixture ranges from about 50% to 80%.

4. The method of claim 1, wherein the mixture further comprises one or more agents selected from the group consisting of a solublizing agent, a structuring agent, a sweetening agent, a taste-masking agent, a coloring agent, a stabilizing agent, a flavoring agent, a plastizer, a surfactant, a saliva stimulating agent, an antibacterial agent, an emulsifying agent, a binding agent, a permeation enhancer, a buffering agent and a pigment.

5. The method of claim 1, wherein the mixture further comprises a solublizing agent selected from PEG400, glycerin, propylene glycol, and triacetin.

6. The method of claim 6, wherein the solublizing agent ranges about 5-25% by weight in the composition.

7. The method of claim 6, wherein the solublizing agent is PEG400.

8. The method of claim 1, wherein the mixture further comprises a structuring agent selected from the group consisting of Pullulan, hydroxypropylmethyl cellulose (HPMC), hydroxypropyl cellulose (HPC), Polyvinylpyrrolidone (PVP), carboxymethyl cellulose (CMC) and its sodium salt, methylcellulose, microcrystalline cellulose, pectin, polyvinyl acetate (PVA), and sodium alginate, modified starch, microcrystalline cellulose, gelatine, polyacrylate, and polyethylene oxide (PEO), and wherein the structuring agent ranges from about 5% to 80% in the composition.

9. The method of claim 1, wherein the pre-designed pattern is configured so that the porous solid dosage composition contains void space ranging between 10% to 80% of the total volume of the composition.

10. The method of claim 9, wherein the void space ranges between 30% to 60% of the total volume of the composition.

11. The method of claim 9, wherein the void space ranges between 20% to 50% of the total volume of the composition.

12. The method of claim 1, wherein the composition has a density of about 15-800 mg/cm³.

13. The method of claim 1, wherein the composition has a density of about 25-200 mg/cm³.

14. The method of claim 1, wherein the pharmaceutically active agent is selected from the group consisting of β-lactam antibiotics, Caffeine, Caffeine salt compounds, vitamins, antibiotics, antacids, analgesics, anti-inflammatory agents, laxatives, anorexics, anti-asthmatics, diuretics, antiflatulents, antimigraine agents, anti-arrhythmic agents, antispasmodics, sedatives, antihyperactive agents, tranquilizers, antihistamines, decongestants, beta-blockers, coronary vasodilators, bronchodilators, muscle relaxants, anticoagulants, antileptic agents, anti-emetics, hypotensives, sympathomimetic agents, expectorants, oral antidiabetic agents, anti-inflammatory and anti-rheumatic agent, tranquilizers, cardiovascular agents and cerebral vasodilators, cerebral protectors, antispasmodic, antisecretory agents, hepatic protectors, hormones, contraceptives, polypeptides and proteins, medicaments intended for the treatment of ADHD, allergies, vaccines, erectile dysfunction, and combinations thereof.

15. The method of claim 1, wherein the pharmaceutically active agent is selected from the group consisting of penicillins, penicillin derivatives, cephalosporins, monobactams, carbapenems, benzathine penicillin, benzylpenicillin (penicillin G), phenoxymethylpenicillin (penicillin V), procaine penicillin, oxacillin, methicillin, nafcillin, cloxacillin, dicloxacillin, flucloxacillin, temocillin, amoxicillin, ampicillin, azlocillin, carbenixillin, mezlocillin, and piperacillin.

16. The method of claim 1, wherein the pharmaceutically active agent is an antibiotic, and the antibiotic ranges from about 5% to about 85% by weight in the composition.

17. The method of claim 1, wherein the pharmaceutically active agent is Amoxicillin.

18. The method of claim 1, wherein the composition disintegrates substantially in the mouth of a subject within 5-10 minutes.

19. The method of claim 1, wherein the water is removed by heating, infra-red, UV, microwave, freeze-drying, direct-ion technology, or a combination thereof.

20. A porous dosage composition prepared by the method of claim 1.
